SEQUENCE LISTING (16 sequences)

SEQ ID NO: 1 - DNA, 101 bp, Solanum lycopersicum
  variation (51)..(51): SNP527; n= A or T
catgtaaatgtgggtcgcttgatcaaatttttagtttcttggagatctganaaacaagggctgtatgagaaaatcttagaattgagccatacaatggcact

SEQ ID NO: 2 - DNA, 101 bp, Solanum lycopersicum
  variation (51)..(51): SNP 001; n= C or G
taatctgtttctggataaatgcctctccaatttcgataaataagggatcgnaaggagataggagaaaagtacaacaccatcgagagtcaccactaacagtg

SEQ ID NO: 3 - DNA, 101 bp, Solanum lycopersicum
  variation (51)..(51): SNP224; n= A or T
gatggttttggtttcgctagccaaatttttaacatcaatcaagcaagagtnaaagcaggcatatttatatctgaggatccaacagtgttcacctcttcttc

SEQ ID NO: 4 - DNA, 101 bp, Solanum lycopersicum
  variation (51)..(51): SNP 540; n= A or G
ccatcagctgatctcaactacccgtcattcattgccttgtacagcattganggaaacttcactttgttggagcagaaattcaagaggacagttacaaatgt

SEQ ID NO: 5 - DNA, 101 bp, Solanum lycopersicum
  variation (51)..(51): SNP 066; n= A or T
cactacaagcctagataacaaaagtatatccttcagtaagcatacatggtntcagccaacttcaaaccccgagacttgaagaatcaatccttcccgaggct

SEQ ID NO: 6 - DNA, 101 bp, Solanum lycopersicum
  variation (51)..(51): SNP 521; n= A or G
aagtatggctggtatcatcgtcgtcgtcgtcccggaagaatccaatatcanaaaaaaagggttcttcttcctcctcctgtaaaattgtcggctgagattga

SEQ ID NO: 7 - DNA, 101 bp, Solanum lycopersicum
  variation (51)..(51): SNP 434; n= A or C
gattatggggacgaggatgcaaacaaaacactagccaagctgtaagtagtntctcatggattatattatacgaacttttaaaaaatattatcgcagagcgt

SEQ ID NO: 8 - DNA, 101 bp, Solanum lycopersicum
  variation (51)..(51): SNP 044; n= T or C
ttaggcatgtactcgtaaaccaaaagatttgtctcatgattcgagcaaaancctaataatctaacaatgtgcctgtgtcggatcctcccaagagtctgtat

SEQ ID NO: 9 - DNA, 101 bp, Solanum lycopersicum
  variation (51)..(51): SNP 017; n= A or G
cacttcaccgacacccatctccttatctacttttctatcgtcacatactcnacggaatttgtatgctttcttagttactgccaaaattgctcttcgaacta

SEQ ID NO: 10 - DNA, 101 bp, Solanum lycopersicum
  variation (51)..(51): SNP 304; n= A or G
cgtcgatcaaaatactcttccaaacctaccgcaaaagataatagcaggcantaacaacaaagattaatctcccccctatatatgacttgagttgtcaggaa

SEQ ID NO: 11 - DNA, 101 bp, Solanum lycopersicum
  variation (51)..(51): SNP 131; n= T or C
aatagaaatggaaattgtactccattttgacgaattttggttgcacattgntagggacttcacaacattgatttgctactcatttttatgtgcccgttact

SEQ ID NO: 12 - DNA, 101 bp, Solanum lycopersicum
  variation (51)..(51): SNP 155; n= T or G
atcctctaatcgttcggccgattcgactcagtcgaaaaatccttttcagtngctgcttccaccgatgcagattcctccaccgccaccaccaccgccgcctc

SEQ ID NO: 13 - DNA, 101 bp, Solanum lycopersicum
  variation (51)..(51): SNP 151; n= T or C
aaggcgaaaattgggaaggaatcattactagaatttggccaaacacaaagnaccttgatgtgatagtcacaggtgcaatggctcaatatatacctactttg

SEQ ID NO: 14 - DNA, 101 bp, Solanum lycopersicum
  variation (51)..(51): SNP 193; n= A or G
attttgtttcttttgattcaatttcagtttcttcttttctccttttcacanaagcaaaaagacagagaacagctgcaatcaagatcaatgttccaactaca

SEQ ID NO: 15 - DNA, 101 bp, Solanum lycopersicum
  variation (51)..(51): SNP 097; n= T or C
ttcttcaattgttggtctgaaatagcgcgcagtggagattccaatcttatngttaataaaccgccggttctggaccgttggtttcctgaatttgttgctag

SEQ ID NO: 16 - DNA, 101 bp, Solanum lycopersicum
  variation (51)..(51): SNP 034; n= A or G
ggtaacaaaatatacgtagaagggggatattacttcgaggggtattgggtngaaatcgtgggaacgtacgataacatcaccgacaggtatttagcaataga

(19) United States
(12) Patent Application Publication
(10) Pub. No.: US 2021/0163981 A1
(43) Pub. Date: Jun. 3, 2021

(54) Tospovirus Resistant Plants and Methods Thereof

(71) Applicants: Tierra Seed Science Pvt. Ltd., Hyderabad (IN); SEQ ID 1.0 INC, Toronto (CA)

(72) Inventors: Parthasarathi Bhattacharya, Hyderabad (IN); Suresh Atluri, Hyderabad (IN); Surinder Kumar Tikoo, Hyderabad (IN); Charles Pick, Toronto (CA)

(21) Appl. No.: 17/162,239

(22) Filed: Jan. 29, 2021

Related U.S. Application Data
(63) Continuation-in-part of application No. 15/555,937, filed on Sep. 5, 2017, now abandoned.

(51) Int. Cl. C12N 15/82; C12Q 1/6895

(52) U.S. Cl. CPC C12N 15/8283; C12Q 2600/156; C12Q 2600/13; C12Q 1/6895

(57) ABSTRACT

The present invention provides tomato plants exhibiting resistance to the TOSPO (GBNV) Virus exhibiting the traits of healthy and evenly ripened fruits without brown spots, reduced wilting of the top portion of the tomato plants and reduced dark spots on the leaves and stems of the tomato plant, and materials useful for improving tomato yield. In particular, the present invention provides a path to the development of hybrid tomato plants exhibiting resistance to TOSPO (GBNV) virus infection, seed of hybrid tomato plants exhibiting TOSPO (GBNV) virus resistance, and methods of producing the same. Methods described herein involve the use of marker-assisted selection and marker-assisted backcrossing utilizing molecular markers associated with a tomato TOSPO Resistance phenotype.

### CROSS-REFERENCE TO RELATED APPLICATIONS

This Application is a continuation-in-part of U.S. patent application Ser. No. 15/555,937, filed on Sep. 5, 2017, which application is a 371 application of PCT/IB2017/055151 filed on Aug. 28, 2017; the disclosures of which applications are herein incorporated by reference.

### INTRODUCTION

### Field of the Invention

The present invention relates to the field of agricultural biotechnology more specifically to the development of disease resistant tomato lines.

### Background

Viruses of the genus TOSPO cause significant worldwide crop losses. Tospoviruses have a tripartite RNA genome of ambisense polarity. The Tospovirus genus includes plant viruses that infect a wide range of plant species including vegetable, fruit, and ornamental crops. The viruses belonging to this genus are popularly described as Tospovirus and the tomato spotted wilt virus is a type of species belonging to this genus. Tospoviruses are distributed in tropical, subtropical and temperate regions throughout the Northern Hemisphere, Western Europe and Asia. Groundnut bud necrosis virus (GBNV) is currently recognized as the most economically important Tospovirus in South Asia & South East Asia. Losses due to GBNV alone have been estimated at more than US$89 million per annum in Asia.

The transmission of the disease is by the vector Thrips palmi. The disease is affecting tomato crop cultivation to the tune of 350,000 hectares per year in India, especially in Kharif season. Control of Tospoviruses remains problematic. Cultural practices and varietal selection have proven effective in minimizing losses due to tomato spotted wilt virus (TSWV) in some field crops. A series of risk factors including prior history, planting date, cultivar selection and plant and row spacing have been identified as critical factors in peanuts. In other high-risk areas, such as Hawaii, highly susceptible crops cannot be grown profitably. In greenhouse grown crops, such as floral crops, extreme measures including screening of production areas with fine-meshed cloth, preventative Thrips management strategies and use of propagation material shown to be free of TSWV and impatiens necrosis spot virus (INSV) are necessary for control of these viruses.

While forms of resistance have been introduced into various crops, they have nearly always been overcome by the rapid occurrence of resistance-breaking strains of the virus. TSWV is thought to exist in nature as a complex heterogeneous mixture of distinct isolates that can exchange genetic information through re-assortment of genome segments. This provides a readily available reservoir of genetic information to facilitate adaptation. For the reasons stated above control of the disease is a challenge so far. Various resistant genes were identified and designated as Swa1, Swb1, Sw2, Sw3, and Sw4, Sw-5, Sw6, Sw7 from wild source of tomato (S. peruvianum) against TOSPO virus.

However, these resistance genes are not effective to control the GBNV disease in India. Chemical control measures have not been able to contain the disease and farmers are suffering yield loss on account of the disease outbreak. The estimated loss (Kunkalikar, S. R. et al (2011) is around 80% per season of the crop. The calculated loss to the farmer in India is approximate US$25 million if approximately 50% of acreage gets affected by the disease i.e. 175,000 hectares.

As known genetic resistance and chemical measures are not effective in the control of the disease, there is a need to develop an edible and cultivable tomato line resistant to GNB V.

### SUMMARY

Aspects of the invention include an edible and cultivable tomato line resistant to GNBV, and related method for developing such a line.

The invention also relates to a cultivable & edible F1 hybrid variety resistant to GBNV, and related method for developing such a hybrid line.

The invention relates to the identification of DNA markers linked to GBNV resistance trait and use of marker-assisted breeding for selection of resistant edible and cultivable tomato line or F1 hybrid variety

### BRIEF DESCRIPTION OF DRAWINGS AND FIGURES

FIG. 1 shows the flow chart of breeding interspecific cross up to BC2F2.

FIG. 2 shows the flow chart of breeding BC2F3 to current status.

FIG. 3 depicts the frequency distribution of TOSPO (GBNV) disease over generations.

FIG. 4 shows photomicrographs of the disease scoring chart.

FIG. 5 depicts the distribution of BC2F3 lines with different recipient allele frequency based on 2455 SNP.

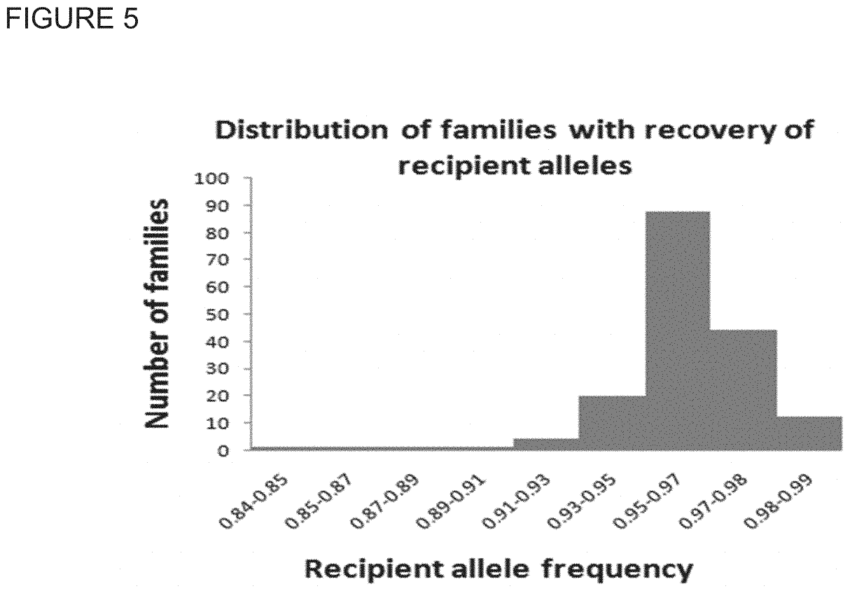

FIG. 6 shows BC2F6 & BC2F7 families (RP×SP1)×SP2 indicating trait linked markers in resistant progenies.

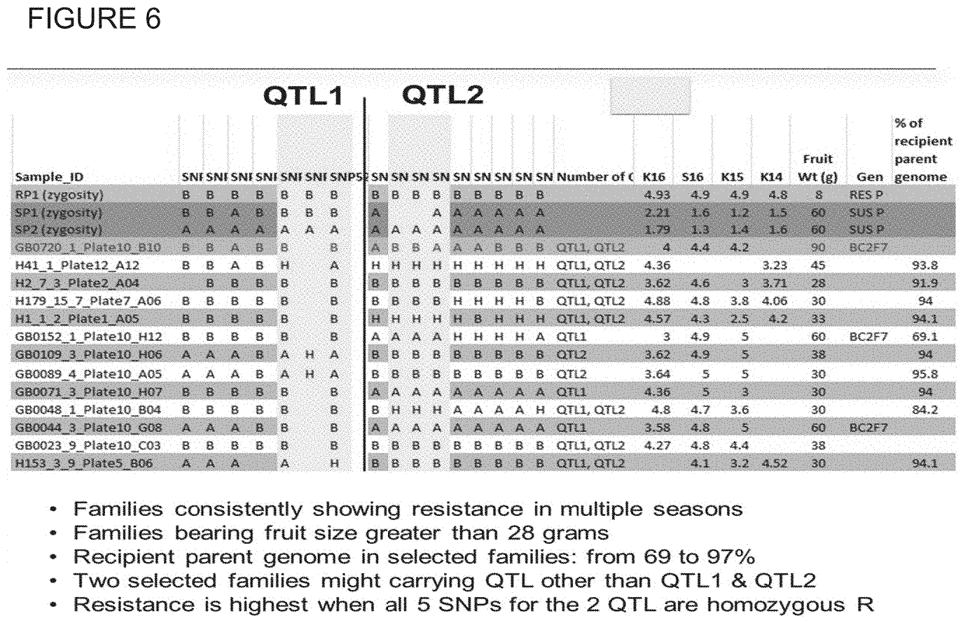

### SEQUENCE LISTING

A computer-readable text file, entitled “FP07311 SequenceListing.txt,” created on or about Jan. 27, 2021 with a file size of about 6 kb contains the sequence listing for this application and is hereby incorporated by reference in its entirety. The Tomato Genotype Data, including assembled tomato genome version 2.40 contained in websites and publications by Solanaceae Coordinated Agricultural Project (SolCAP) is incorporated by reference in its entirety. The tomato genome was published on May 30, 2021 at The Tomato Genome Consortium., Kazusa DNA Research Institute., Sato, S. et al. The tomato genome sequence provides insights into fleshy fruit evolution. Nature 485, 635-641 (2012). https://doi.org/10.1038/nature11119; the disclosure of which is herein incorporated by reference.

### DETAILED DESCRIPTION

The inventors have identified Solanum peruvianum line which displays tolerance or resistance to Ground nut bud necrosis virus (GBNV). The inventors have identified the Single Nucleotide Polymorphisms (SNPs) which confers the GBNV-resistance trait. Further, the inventors have successfully been able to introgress the trait from the donor Solanum peruvianum in Solanum lycopersicum lines through marker assisted breeding. This has led to the development of cultivable and edible variety of Solanum lycopersicum which are resistant to GBNV.

Accordingly, the present invention provides an edible and cultivable tomato line resistant to Ground nut bud necrosis virus (GBNV), and related method for developing such line.

The invention also relates to development of cultivable and edible F1 hybrid variety resistant to GBNV, and related method thereof.

The invention relates to the identification of markers and use of marker assisted breeding for selection of GBNV resistant edible and cultivable tomato line.

The donor is a Solanum peruvianum line obtained from World Vegetable Centre—Asian Vegetable Research and Development Center (AVRDC), Taiwan. The donor line is commercially available, and it is also available from the Asian Vegetable Research and Development Center (AVRDC), Taiwan under Accession No. L00671.

Cultivated lines used for development of progeny were SP-1 (Susceptible parent 1—Sel 22, obtained from Indian Institute of Horticultural Research Bengaluru (IIHR), a commercial variety since 1985) and SP-2 (Susceptible line)—collected from AVRDC carrying Ty 2 gene that gives partial resistance against TyLCV (Tomato Yellow Leaf Curl Virus) but susceptible to TOSPO (GBNV).

In an embodiment, any suitable susceptible line of Solanum lycopersicum can be used for development of the progeny having resistance to GBNV. Preferably, a S. lycopersicum recipient plant according to the invention is a commercial plant or line.

The term “Resistance” as used herein is as defined by the ISF (International Seed Federation) Vegetable and Ornamental Crops Section for describing the reaction of plants to pests or pathogens, and abiotic stresses for the Vegetable Seed Industry.

The term “single nucleotide polymorphism” or “SNP” means substitution of a deoxynucleotide in a single stranded DNA sequence by a different deoxynucleotide. Such substitution is commonly referred to as single base substitution, where the bases are adenine, guanine, cytosine, and thymine. The respective deoxynucleotides are deoxyadenylate, deoxyguanylate, deoxycytidylate, and deoxythymidylate. The bases and the deoxynucleotides are represented as A, G, C, and T respectively. When a SNP is linked to a specific trait (such as resistance to GBNV), the deoxynucleotide that is substituted is considered the SNP.

The term “variant” refers to a single nucleotide polymorphism that is different than its wild type counterpart, and which can confer the GBNV-resistance trait.

The term “The term “quantitative trait locus” or “QTL” refers to a polymorphic genetic locus with at least two alleles that differentially affect the expression of a phenotypic trait in at least one genetic background, e.g., in at least one breeding population or progeny.

In one embodiment, the present invention discloses molecular genetic markers, especially SNPs, linked to the GBNV resistance loci.

In one embodiment, the invention provides a method of producing a tomato plant, wherein the tomato plant exhibits resistance to Groundnut bud necrosis virus infection, comprising:
i) providing a donor Solanum peruvianum tomato plant exhibiting resistance to Groundnut bud necrosis virus infection, wherein the donor plant comprises at least one SNP corresponding to a quantitative trait locus (QTL) indicative of Groundnut bud necrosis virus resistance on chromosome 1 or chromosome 2, and wherein the SNP is selected from a group consisting of:
   (a) variant A or T of SNP solcap_snp_sl_1815 at position 74315042 of chromosome 1 on the tomato genome version 2.40 (SEQ ID NO: 1);
   (b) variant C or G of SNP solcap_snp_sl_1819 at position 74329911 of chromosome 1 on the tomato genome version 2.40 (SEQ ID NO: 2);
   (c) variant A or T of SNP solcap_snp_sl_1824 at position 74464953 of chromosome 1 on the tomato genome version 2.40 (SEQ ID NO: 3);
   (d) variant A or G of SNP solcap_snp_sl_17075 at position 74483269 of chromosome 1 on the tomato genome version 2.40 (SEQ ID NO: 4);
   (e) variant A or T of SNP solcap_snp_sl_1827 at position 74602284 of chromosome 1 on the tomato genome version 2.40 (SEQ ID NO: 5);
   (f) variant A or G of SNP solcap_snp_sl_21280 at position 76783695 of chromosome 1 on the tomato genome version 2.40 (SEQ ID NO: 6);
   (g) variant A or C of SNP solcap_snp_sl_34568 at position 76905658 of chromosome 1 on the tomato genome version 2.40 (SEQ ID NO: 7);
   (h) variant T or C of SNP solcap_snp_sl_36202 at position 47651366 of chromosome 2 on the tomato genome version 2.40 (SEQ ID NO: 8);
   (i) variant A or G of SNP solcap_snp_sl_21866 at position 47950096 of chromosome 2 on the tomato genome version 2.40 (SEQ ID NO: 9);
   (j) variant A or G of SNP solcap_snp_sl_21862 at position 47981517 of chromosome 2 on the tomato genome version 2.40 (SEQ ID NO: 10);
   (k) variant T or C of SNP solcap_snp_sl_31689 at position 47996412 of chromosome 2 on the tomato genome version 2.40 (SEQ ID NO: 11);
   (l) variant T or G of SNP solcap_snp_sl_20063 at position 48455087 of chromosome 2 on the tomato genome version 2.40 (SEQ ID NO: 12);
   (m) variant T or C of SNP solcap_snp_sl_21966 at position 48370810 of chromosome 2 on the tomato genome version 2.40 (SEQ ID NO: 13);
   (n) variant A or G of SNP solcap_snp_sl_58447 at position 48602171 of chromosome 2 on the tomato genome version 2.40 (SEQ ID NO: 14);
   (o) variant T or C of SNP solcap_snp_sl_20049 at position 48697164 of chromosome 2 on the tomato genome version 2.40 (SEQ ID NO: 15); and
   (p) variant A or G of SNP solcap_snp_sl_21867 at position 47948927 of chromosome 2 on the tomato genome version 2.40 (SEQ ID NO: 16);
ii) crossing the donor tomato plant with a recipient Solanum lycopersicum plant to produce F1 Solanum lycopersicum progeny tomato plants, wherein the recipient tomato plants does not exhibit resistance to Groundnut bud necrosis virus and the crossing results in introgression of one or more quantitative trait locus (QTL) on chromosome 1 or chromosome 2 indicative of Groundnut bud necrosis virus resistance in the progeny tomato plants;
iii) generating further offspring progeny tomato plants from said F1 progeny plant by backcrossing or selfing; and
iv) identifying and selecting a Groundnut bud necrosis virus resistant progeny tomato plant obtained through step (b) and step (c), wherein the progeny tomato plant comprises at least one SNP corresponding to a quantitative trait locus (QTL) indicative of Groundnut bud necrosis virus resistance on chromosome 1 or chromosome 2, and wherein the SNP is selected from a group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.
In one embodiment, the introgressed sequences are preferably found at one or more of the loci defined by the following SNP markers:
   (a) variant A of SNP solcap_snp_sl_1815 at position 74315042 of chromosome 1 on the tomato genome version 2.40 (SEQ ID NO: 1);
   (b) variant T of SNP solcap_snp_sl_1815 at position 74315042 of chromosome 1 on the tomato genome version 2.40 (SEQ ID NO: 1);
   (c) variant C of SNP solcap_snp_sl_1819 at position 74329911 of chromosome 1 on the tomato genome version 2.40 (SEQ ID NO: 2);
   (d) variant G of SNP solcap_snp_sl_1819 at position 74329911 of chromosome 1 on the tomato genome version 2.40 (SEQ ID NO: 2);
   (e) variant A of SNP solcap_snp_sl_1824 at position 74464953 of chromosome 1 on the tomato genome version 2.40 (SEQ ID NO: 3);
   (f) variant T of SNP solcap_snp_sl_1824 at position 74464953 of chromosome 1 on the tomato genome version 2.40 (SEQ ID NO: 3);
   (g) variant A of SNP solcap_snp_sl_17075 at position 74483269 of chromosome 1 on the tomato genome version 2.40 (SEQ ID NO: 4);
   (h) variant G of SNP solcap_snp_sl_17075 at position 74483269 of chromosome 1 on the tomato genome version 2.40 (SEQ ID NO: 4);
   (i) variant A of SNP solcap_snp_sl_1827 at position 74602284 of chromosome 1 on the tomato genome version 2.40 (SEQ ID NO: 5);
   (j) variant T of SNP solcap_snp_sl_1827 at position 74602284 of chromosome 1 on the tomato genome version 2.40 (SEQ ID NO: 5);
   (k) variant A of SNP solcap_snp_sl_21280 at position 76783695 of chromosome 1 on the tomato genome version 2.40 (SEQ ID NO: 6);
   (l) variant G of SNP solcap_snp_sl_21280 at position 76783695 of chromosome 1 on the tomato genome version 2.40 (SEQ ID NO: 6);
   (m) variant A of SNP solcap_snp_sl_34568 at position 76905658 of chromosome 1 on the tomato genome version 2.40 (SEQ ID NO: 7);
   (n) variant C of SNP solcap_snp_sl_34568 at position 76905658 of chromosome 1 on the tomato genome version 2.40 (SEQ ID NO: 7);
   (o) variant T of SNP solcap_snp_sl_36202 at position 47651366 of chromosome 2 on the tomato genome version 2.40 (SEQ ID NO: 8);
   (p) variant C of SNP solcap_snp_sl_36202 at position 47651366 of chromosome 2 on the tomato genome version 2.40 (SEQ ID NO: 8);
   (q) variant A of SNP solcap_snp_sl_21866 at position 47950096 of chromosome 2 on the tomato genome version 2.40 (SEQ ID NO: 9);
   (r) variant G of SNP solcap_snp_sl_21866 at position 47950096 of chromosome 2 on the tomato genome version 2.40 (SEQ ID NO: 9);
   (s) variant A of SNP solcap_snp_sl_21862 at position 47981517 of chromosome 2 on the tomato genome version 2.40 (SEQ ID NO: 10);
   (t) variant G of SNP solcap_snp_sl_21862 at position 47981517 of chromosome 2 on the tomato genome version 2.40 (SEQ ID NO: 10);
   (u) variant T of SNP solcap_snp_sl_31689 at position 47996412 of chromosome 2 on the tomato genome version 2.40 (SEQ ID NO: 11);
   (v) variant C of SNP solcap_snp_sl_31689 at position 47996412 of chromosome 2 on the tomato genome version 2.40 (SEQ ID NO: 11);
   (w) variant T of SNP solcap_snp_sl_20063 at position 48455087 of chromosome 2 on the tomato genome version 2.40 (SEQ ID NO: 12);
   (x) variant G of SNP solcap_snp_sl_20063 at position 48455087 of chromosome 2 on the tomato genome version 2.40 (SEQ ID NO: 12);
   (y) variant T of SNP solcap_snp_sl_21966 at position 48370810 of chromosome 2 on the tomato genome version 2.40 (SEQ ID NO: 13);
   (z) variant C of SNP solcap_snp_sl_21966 at position 48370810 of chromosome 2 on the tomato genome version 2.40 (SEQ ID NO: 13);
   (aa) variant A of SNP solcap_snp_sl_58447 at position 48602171 of chromosome 2 on the tomato genome version 2.40 (SEQ ID NO: 14);
   (bb) variant G of SNP solcap_snp_sl_58447 at position 48602171 of chromosome 2 on the tomato genome version 2.40 (SEQ ID NO: 14);
   (cc) variant T of SNP solcap_snp_sl_20049 at position 48697164 of chromosome 2 on the tomato genome version 2.40 (SEQ ID NO: 15); and
   (dd) variant C of SNP solcap_snp_sl_20049 at position 48697164 of chromosome 2 on the tomato genome version 2.40 (SEQ ID NO: 15); and
   (ee) variant A of SNP solcap_snp_sl_21867 at position 47948927 of chromosome 2 on the tomato genome version 2.40 (SEQ ID NO: 16);
   (ff) variant G of SNP solcap_snp_sl_21867 at position 47948927 of chromosome 2 on the tomato genome version 2.40 (SEQ ID NO: 16).

Insofar as the introgressed sequences from Solanum peruvianum conferring resistance to GBNV can be marked by the specific alleles of the SNP markers of the invention, a plant of the invention has at least one of the alleles or variants as defined by the embodiments of this invention. Preferably, the SNPs detailed for the embodiments of the present invention are used as markers for the detection of introgressed sequence from Solanum peruvianum.

In another embodiment, the invention provides a method for producing the tomato plant, wherein SNPs present on chromosome 1 are indicated by SEQ ID NO: 1, SEQ ID NO: 2, SEQ ID NO: 3, SEQ ID NO: 4, SEQ ID NO: 5, SEQ ID NO: 6 and SEQ ID NO: 7 and SNPs present on chromosome 2 are indicated by SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

In another embodiment, the invention provides a method for producing the tomato plant of claim 1, wherein the progeny tomato plant does not exhibit one or more trait indicative of Groundnut bud necrosis virus infection selected from a group consisting of deformed shape, uneven ripening, yellow patch formation on the fruit, wilting of the top portion of the plant, dark brown spots on the leaves and dark brown patches on stems of the plants. Some of the post infection common characteristics traits of the recipient tomato plant which is susceptible to TOSPO are deformed shape and uneven ripening of the fruit, wilting of the top portion of the plant, dark brown spots on the leaves and dark brown patches on stems of the plants.

In another embodiment, the progeny plant is selected at each stage on the basis of one or more of the alleles or variants of the SNP markers as defined in the embodiments of the present invention.

In another embodiment, the SNP markers according to the embodiment of the invention are marker loci linked to chromosomal regions or QTL that are involved in or associated with the GBNV tolerance or resistance phenotype. The allele or variants of these markers thus indicates whether the sequences surrounding the markers are introgressed from Solanum peruvianum or not, introgressed sequences at this locus being correlated to resistance or tolerance to Groundnut Bud Necrosis Virus (GBNV).

Regarding the QTL or chromosomal regions marked by the SNPs of the invention and correlated with the phenotype, a single of this SNP marker may impart the desired phenotype.

The transfer of one or more nucleic acids is performed by crossing the donor tomato plant with a recipient tomato plant to produce progeny tomato plants comprising at least one QTL for TOSPO virus resistance as in introgression, and wherein identification and selection is performed on one or more progeny plants.

The method of transfer of one or more nucleic acids is based on selecting the TOSPO (GBNV) resistant plants from progenies of an interspecific cross. The crossing was made possible using embryo rescue technique to overcome incompatibility.

Further, the method of the invention is performed by detecting the marker in DNA isolated from the recipient tomato plants or from one or more of progeny plants.

In one embodiment, the extracted or isolated DNA was used for conducting single nucleotide polymorphism analysis based on the Solanaceae Coordinated Agricultural Project (SolCAP). Solcap (Solanaceae Coordinated Agricultural Project) was supported by the Agriculture and Food Research Initiative Applied Plant Genomics CAP Program of USDA's National Institute of Food and Agriculture which led to the development of the Solcap Infinium SNP panel for tomato. This panel is an SNP array that is publicly available and contains information regarding SNPs in tomato. SolCAP is a commonly used tool in tomato genome research to identify polymorphic SNPs in wild or cultivated tomato germplasm.

In another embodiment, SolCAP Infinium genotyping SNP panel has been used for identifying the SNPs associated with the resistance to Groundnut Bud Necrosis virus. The SNPs indicated in the SolCAP Infinium SNP panel is on the basis of chromosome positions as indicated in the tomato genome version SL 2.40. The tomato genotype data, including genome version 2.40 is publicly available on various websites (such as http://solcap.msu.edu/).

The method of the invention comprises selecting a recipient tomato plant that comprises within its genome at least two QTLs selected from the group consisting of QTL1, QTL2 and virus-resistance related parts thereof.

The method of the invention comprises selecting a recipient tomato plant that comprises QTL1 and QTL2 within its genome.

The method of the invention comprises selecting a recipient tomato plant that comprises within its genome a QTL or a viral resistance related part thereof, and at least one QTL selected from the group consisting of QTL1, QTL2 and related parts thereof.

The method of the invention further comprises a step of crossing a recipient tomato plant with a plant of a cultivated variety of tomato to produce progeny plants.

The method of the invention comprises the recipient tomato plant as same as that of the plant of the cultivated variety of tomato to which the recipient tomato plant is crossed.

The resistance provided against by the method of the invention is for a Groundnut Bud Necrosis Virus (GBNV) which is a TOSPO virus.

The invention is for a tomato plant exhibiting TOSPO virus resistance, and the said tomato plant or part thereof comprises a QTL selected from the group consisting of QTL1, QTL2 and TOSPO virus-related parts thereof, wherein the QTLs or viral resistance-related parts thereof are not present in the natural genetic background of the edible tomato plants.

The tomato plant exhibiting TOSPO virus resistance, is produced by backcrossing the tomato plant BC 1 (back cross 1) with a tomato plant that exhibits commercially desirable characteristics, wherein the resulting hybrid plant comprises at least one QTL selected from the group consisting of QTL1, QTL2 and TOSPO virus resistance-related parts thereof, wherein at least one QTL is not present in the natural background of either the tomato plant selected in step 1 (c) or the tomato plant exhibiting commercially desirable characteristics.

Standard embryo rescue method was employed to overcome sexual incompatibility between S. peruvianum & S. lycopersicum, and to raise the seven day old fertilized ovules on tissue culture medium.

In another embodiment, the invention provides a method of identifying a tomato plant resistant to Groundnut bud necrosis virus infection, comprising identifying through genotyping in a nucleic acid sample of the tomato plant at least one SNP selected from a group consisting of:
(a) variant A or T of SNP solcap_snp_sl_1815 at position 74315042 of chromosome 1 on the tomato genome version 2.40 (SEQ ID NO: 1);
(b) variant C or G of SNP solcap_snp_sl_1819 at position 74329911 of chromosome 1 on the tomato genome version 2.40 (SEQ ID NO: 2);
(c) variant A or T of SNP solcap_snp_sl_1824 at position 74464953 of chromosome 1 on the tomato genome version 2.40 (SEQ ID NO: 3);
(d) variant A or G of SNP solcap_snp_sl_17075 at position 74483269 of chromosome 1 on the tomato genome version 2.40 (SEQ ID NO: 4);
(e) variant A or T of SNP solcap_snp_sl_1827 at position 74602284 of chromosome 1 on the tomato genome version 2.40 (SEQ ID NO: 5);
(f) variant A or G of SNP solcap_snp_sl_21280 at position 76783695 of chromosome 1 on the tomato genome version 2.40 (SEQ ID NO: 6);
(g) variant A or C of SNP solcap_snp_sl_34568 at position 76905658 of chromosome 1 on the tomato genome version 2.40 (SEQ ID NO: 7);
(h) variant T or C of SNP solcap_snp_sl_36202 at position 47651366 of chromosome 2 on the tomato genome version 2.40 (SEQ ID NO: 8);
(i) variant A or G of SNP solcap_snp_sl_21866 at position 47950096 of chromosome 2 on the tomato genome version 2.40 (SEQ ID NO: 9);
(j) variant A or G of SNP solcap_snp_sl_21862 at position 47981517 of chromosome 2 on the tomato genome version 2.40 (SEQ ID NO: 10);
(k) variant T or C of SNP solcap_snp_sl_31689 at position 47996412 of chromosome 2 on the tomato genome version 2.40 (SEQ ID NO: 11);
(l) variant T or G of SNP solcap_snp_sl_20063 at position 48455087 of chromosome 2 on the tomato genome version 2.40 (SEQ ID NO: 12);
(m) variant T or C of SNP solcap_snp_sl_21966 at position 48370810 of chromosome 2 on the tomato genome version 2.40 (SEQ ID NO: 13);
(n) variant A or G of SNP solcap_snp_sl_58447 at position 48602171 of chromosome 2 on the tomato genome version 2.40 (SEQ ID NO: 14);
(o) variant T or C of SNP solcap_snp_sl_20049 at position 48697164 of chromosome 2 on the tomato genome version 2.40 (SEQ ID NO: 15); and
(p) variant A or G of SNP solcap_snp_sl_21867 at position 47948927 of chromosome 2 on the tomato genome version 2.40 (SEQ ID NO: 16);
wherein the presence of one or more SNP is indicative of Groundnut bud necrosis virus infection resistance trait of the tomato plant.

In another embodiment, the invention provides a method of selecting a tomato plant, wherein SNPs present on chromosome 1 are indicated by SEQ ID NO: 1, SEQ ID NO: 2, SEQ ID NO: 3, SEQ ID NO: 4, SEQ ID NO: 5, SEQ ID NO: 6 and SEQ ID NO: 7 and SNPs present on chromosome 2 are indicated by SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

In another embodiment, the invention provides a tomato plant exhibiting resistance to Groundnut bud necrosis virus infection produced, wherein the tomato plant comprises at least one SNP corresponding to a quantitative trait locus on chromosome 1 or chromosome 2, and wherein the SNP is selected from a group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

In another embodiment, the invention provides a tomato plant, wherein SNPs present on chromosome 1 are indicated by SEQ ID NO: 1, SEQ ID NO: 2, SEQ ID NO: 3, SEQ ID NO: 4, SEQ ID NO: 5, SEQ ID NO: 6 and SEQ ID NO: 7 and SNPs present on chromosome 2 are indicated by SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

In another embodiment, the invention provides a seed of the tomato plant produced by the process as described herein.

Scoring of the disease symptoms (phenotyping) was performed in a hot spot location for TOSPO (GBNV) disease and were categorized into 5 types (1) whole plant diseased (score 1); (2) diseased with few green stem and leaves (score 2); (3) 50% plant covered-may have 1 or 2 fruit (score 3); (4) Except for the top leaves, stem and other plant parts are healthy (score 4); (5) Healthy plants (score 5) using the phenotypic symptoms observed in the field at young and adult plant stages, e.g., 40, 60, 80 days after transplantation in the field.

### EXAMPLES

The following examples are for the purpose of illustration of the invention and are not intended in any way to limit the scope of the invention.

### Materials:

Solanum peruvianum line was obtained from World Vegetable Centre—Asian Vegetable Research and Development Center (AVRDC), Taiwan. The donor line is commercially available, and it is also available from the Asian Vegetable Research and Development Center (AVRDC), Taiwan under Accession No. L00671.

The cultivated lines used were SP-1 (Susceptible parent 1—Sel 22, purchased from Indian Institute of Horticultural Research Bengaluru (IIHR), a commercial line since 1985). SP-2 (Susceptible line)—A Ty 2 line collected from AVRDC but susceptible to TOSPO.

### Example 1

### Development and Determination of Disease Resistant Phenotype

The Solanum peruvianum accession L00671 was used as donor. The S. peruvianum accession L00671 from AVRDC is resistant to Ground nut bud necrosis virus. The donor was crossed with a GBNV susceptible S. lycopersicum line (SP1—that responds well to tissue culture).

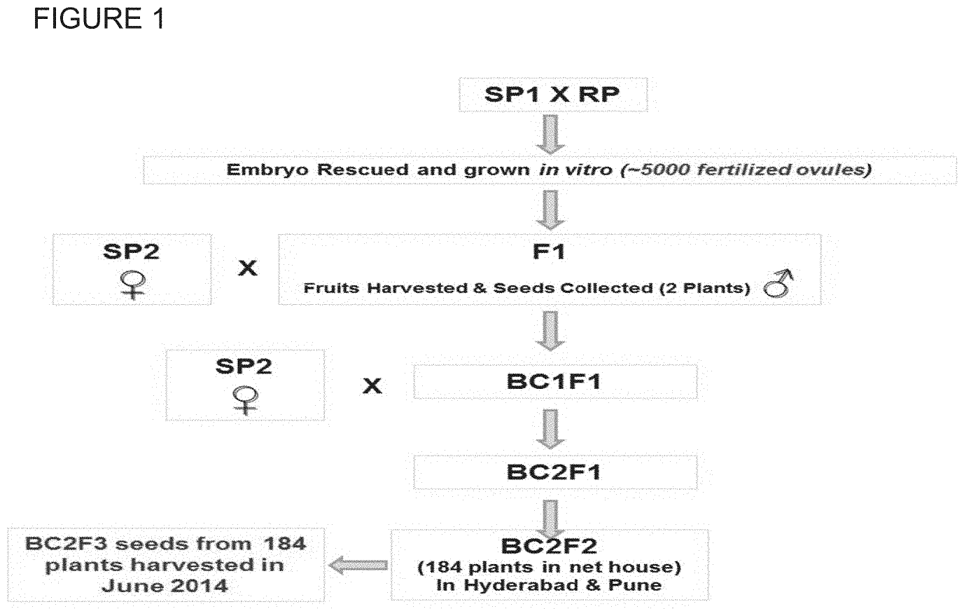

To overcome sexual incompatibility between S. peruvianum & S. lycopersicum, standard embryo rescue method was employed to raise the seven day old fertilized ovules on (FIG. 1 detailing the crossing and backcrossing) tissue culture medium.

More than 5000 ovules were subjected to embryo rescue method and only two embryos were able to develop into true hybrid plants (F1) (seen phenotypically).

These F1s were crossed with another GBNV susceptible line of S. lycopersicum as it was agronomically better & had more firm fruits (SP2—Susceptible parent 2 (Square Round)) and obtained BC1 seeds. The crossed ovules from these BC1 pollinations, using the inter-specific F1 as pollen source, were subjected to embryo rescue to get BC1 plants that were grown to fruiting.

BC1F1 progeny was grown in the field in rainy season under high Thrips pressure.

Pollen from plants, that showed no GBNV symptoms were used to pollinate the SP1 recipient parent to obtain BC2F1 seeds.

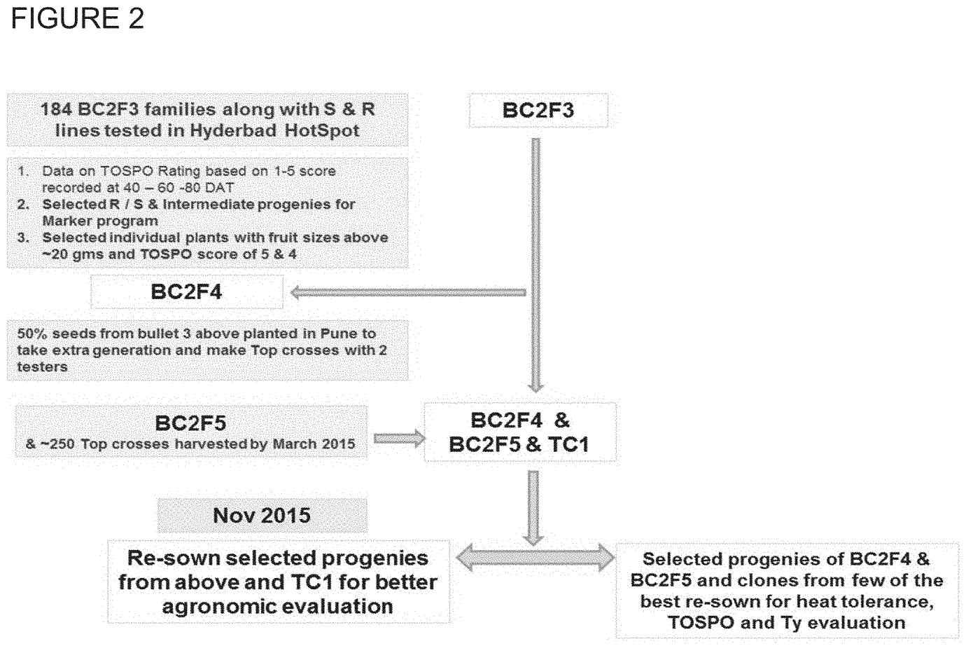

BC2F1 plants were grown and selfed to obtained BC2F2 seeds. Selected BC2F2 plants were grown to obtain BC2F3 (FIG. 2).

Selected BC2F3 families were planted in hot spot location along with the parents (donor as well as recipient) in an augmented design where a susceptible check variety was planted repeatedly after every 10 rows in each bed.

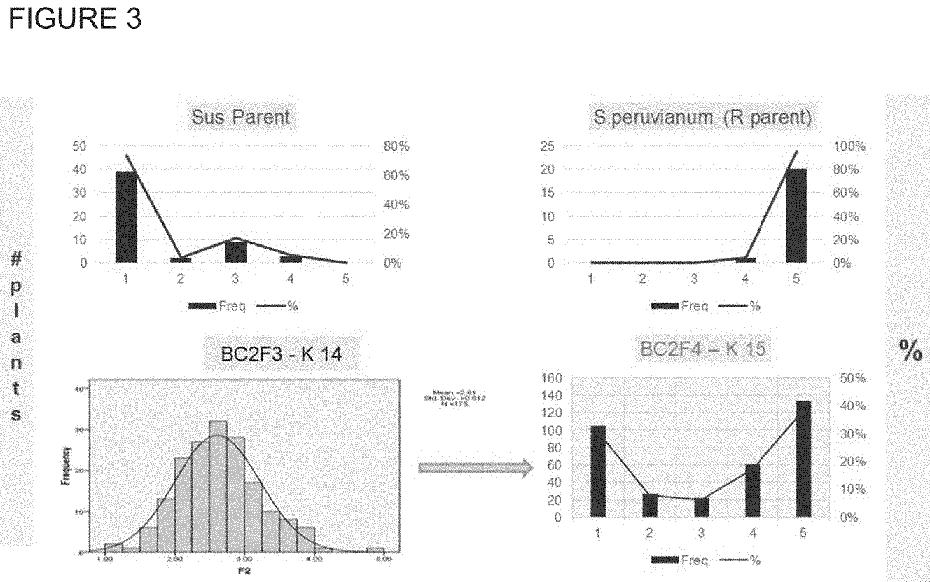

Frequency distribution of the disease in various generations and resistant (RP) and susceptible (SP) parent shown in FIG. 3.

### Example 2

### Development of Field Screening for Disease and Disease Rating Scores

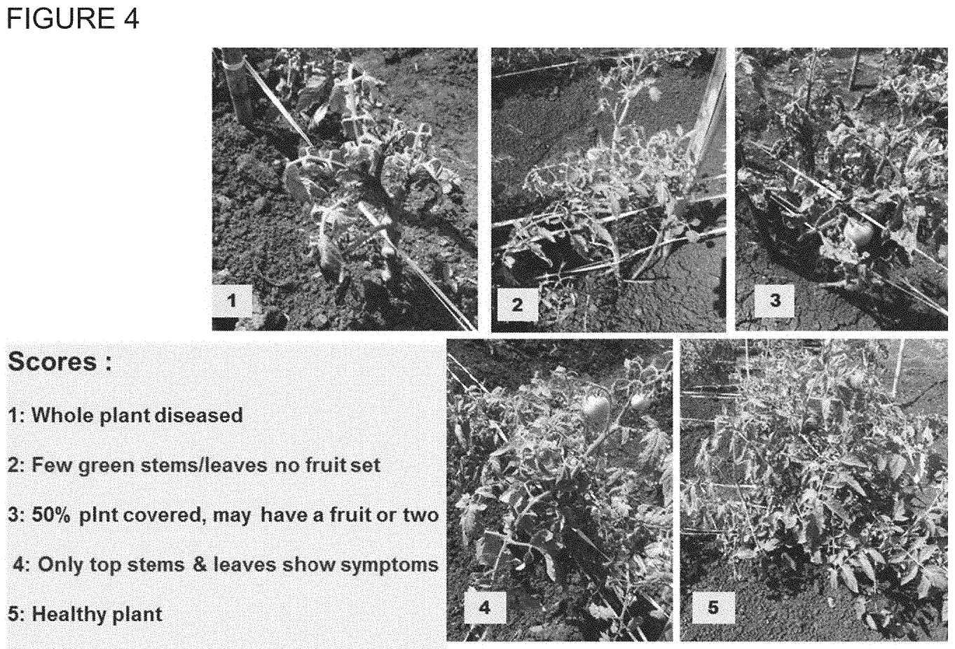

Using the phenotypic symptoms observed in the field at young and adult plant stages, e.g., 40, 60, 80 days after transplantation in the field, the disease symptoms were categorized into 5 types as in FIG. 4 as (1) whole plant diseased (score 1); (2) diseased with few green stem and leaves (score 2); (3) 50% plant covered-may have one or two fruit (score 3); (4) Except for the top leaves, stem and other parts healthy (score 4); (5) Healthy plants (score 5).

Based on data on GBNV scores at 40, 60 & 80 days after transplanting (DAT) the families were grouped into three categories of: 1) Resistant (scores 4, 5); 2) intermediates (score 3) and 3) susceptible (scores 1 and 2). Three individual plants per category were harvested for obtaining BC2F4 seeds. BC2F4 families were again raised and BC2F5 progenies harvested from same groups of families.

The frequency distribution for disease score 1 to 5 for GBNV incidence on resistant parent, susceptible recipient, BC2F3 & BC2F4 progenies are provided in FIG. 3. Disease pressure during the screening has been very high as can be seen by the over 65 percent of susceptible parent shows susceptibility rating of 1 and 2 and another 15 percent having score of 3.

The resistant parent on the other hand showed 90 percent plants scoring rating 5 and another 10 percent scoring a rating of 4, confirming high resistance in this parent (S. peruvianum). BC2F3 distribution for the disease rating shows a normal distribution (FIG. 3: bottom left) indicative of an incomplete dominance for resistance and involvement of more than one gene for its inheritance.

BC2 F4 progenies derived from the susceptible and resistant families of BC2F3 showed the population moving towards resistant & susceptible groups (FIG. 3: bottom right). This indicates that it was possible to select high resistant genotypes with a disease rating scale of 4 & 5.

The mean disease ratings and their standard deviation in case of the BC2F3, BC2F4 & BC2F5 families versus resistant & susceptible parents as well as intermediates are presented in FIG. 3 below.

These data represents three seasons of screening. BC2F5 progenies show an overall stable resistance which is closer to the donor parent while as the susceptible (recipient parent) and a super susceptible line as well as the progenies of intermediate resistance show clear susceptibility over all three seasons of testing. The BC2F5 progenies have further planted and the top stable lines for resistance to GBNV has been selected.

### Example 3

### Genotyping, Linkage and OTL Analysis—Identification of Trait Linked Molecular Markers

To understand precisely the location of resistance genes, 181 leaf samples consisting of 174 BC2F3 families (bulks of 4-20 plants) and 3 parental lines were used. DNA was extracted from each sample using standard extraction method.

### For Marker Identification SNP Panel was Used

Solcap Infinium SNP panel
6,007 SNPs resulted in genotypes with <10% missing datapoints
1,767 showed segregation in the population
556 SNPs selected for linkage construction and QTL analysis based on: Missing datapoints <20%
SNPs were observed to be polymorphic between SP2 and RP1 (original parents)

Trait linked molecular markers by way of multiple QTLs in specified chromosomes is identified. 7000 SNPs were generated and used to screen and identify the SNPs that were closely linked with the resistant trait. Out of 7000 SNPs that were used for the screening on the segregating materials, 16 SNPs were found to be associated with the resistance to GBNV trait.

The screening was done by using Illumina SolCAP Tomato genotyping panel (developed by Illumina, USA). The SolCAP Tomato genotyping panel uses tomato genome sequence version 2.40. The details and the location of the SNPs as indicated in the SolCAP Tomato genotyping panel is indicated in Table 1 along with their sequences.

TABLE 1
Details of SNPs as indicated on SolCAP Infinium SNP panel based on
tomato genome version SL 2.40

| | Chromosome | | | | SEQ ID |
|---|---|---|---|---|---|
| SNP | No | Sequence | SNP | Position | NO |
| QTL 1 | | | | | |
| SNP527 | 1 | CATGTAAATGTGGGTCGCTTGAT | [A/T] | 74315042 | SEQ ID |
| (solcap_ | | CAAATTTTTAGTTTCTTGGAGAT | | | NO: 1 |
| snp_sl_ | | CTGA[A/T]AAACAAGGGCTGTAT | | | |
| 1815) | | GAGAAAATCTTAGAATTGAGCC | | | |
| | | ATACAATGGCACT | | | |
| SNP001 | 1 | TAATCTGTTTCTGGATAAATGCC | [C/G] | 74329911 | SEQ ID |
| (solcap_ | | TCTCCAATTTCGATAAATAAGGG | | | NO: 2 |
| snp_sl_ | | ATCG[C/G[AAGGAGATAGGAGA | | | |
| 1819) | | AAAGTACAACACCATCGAGAGT | | | |
| | | CACCACTAACAGTG | | | |
| SNP224 | 1 | GATGGTTTTGGTTTCGCTAGCCA | [A/T] | 74464953 | SEQ ID |
| (solcap_ | | AATTTTTAACATCAATCAAGCAA | | | NO: 3 |
| snp_sl_ | | GAGT[A/T]AAAGCAGGCATATTT | | | |
| 1824) | | ATATCTGAGGATCCAACAGTGTT | | | |
| | | CACCTCTTCTTC | | | |
| SNP540 | 1 | CCATCAGCTGATCTCAACTACCC | [A/G] | 74483269 | SEQ ID |
| (solcap_ | | GTCATTCATTGCCTTGTACAGCA | | | NO: 4 |
| snp_sl_ | | TTGA[A/G[GGAAACTTCACTTTG | | | |
| 17075) | | TTGGAGCAGAAATTCAAGAGGA | | | |
| | | CAGTTACAAATGT | | | |
| SNP066 | 1 | CACTACAAGCCTAGATAACAAA | [A/T] | 74602284 | SEQ ID |
| (solcap_ | | AGTATATCCTTCAGTAAGCATAC | | | NO: 5 |
| snp_sl_ | | ATGGT[A/T]TCAGCCAACTTCAA | | | |
| 1827) | | ACCCCGAGACTTGAAGAATCAA | | | |
| | | TCCTTCCCGAGGCT | | | |
| SNP521 | 1 | AAGTATGGCTGGTATCATCGTCG | [A/G] | 76783695 | SEQ ID |
| (solcap_ | | TCGTCGTCCCGGAAGAATCCAAT | | | NO: 6 |
| snp_sl_ | | ATCA[A/G]AAAAAAAGGGTTCTT | | | |
| 21280) | | CTTCCTCCTCCTGTAAAATTGTC | | | |
| | | GGCTGAGATTGA | | | |
| SNP434 | 1 | GATTATGGGGACGAGGATGCAA | [A/C] | 76905658 | SEQ ID |
| (solcap_ | | ACAAAACACTAGCCAAGCTGTA | | | NO: 7 |
| snp_sl_ | | AGTAGT[A/C]TCTCATGGATTAT | | | |
| 34568) | | ATTATACGAACTTTTAAAAAATA | | | |
| | | TTATCGCAGAGCGT | | | |
| QTL2 | | | | | |
| SNP044 | 2 | TTAGGCATGTACTCGTAAACCAA | [T/C] | 47651366 | SEQ ID |
| (solcap_ | | AAGATTTGTCTCATGATTCGAGC | | | NO: 8 |
| snp_sl_ | | AAAA[T/C]CCTAATAATCTAACA | | | |
| 36202) | | ATGTGCCTGTGTCGGATCCTCCC | | | |
| | | AAGAGTCTGTAT | | | |
| SNP017 | 2 | CACTTCACCGACACCCATCTCCT | [A/G] | 47950096 | SEQ ID |
| (solcap_ | | TATCTACTTTTCTATCGTCACAT | | | NO: 9 |
| snp_sl_ | | ACTC[A/G]ACGGAATTTGTATGC | | | |
| 21866) | | TTTCTTAGTTACTGCCAAAATTG | | | |
| | | CTCTTCGAACTA | | | |
| SNP304 | 2 | CGTCGATCAAAATACTCTTCCAA | [A/G] | 47981517 | SEQ ID |
| (solcap_ | | ACCTACCGCAAAAGATAATAGC | | | NO: 10 |
| snp_sl_ | | AGGCA[A/G]TAACAACAAAGATT | | | |
| 21862) | | AATCTCCCCCCTATATATGACTT | | | |
| | | GAGTTGTCAGGAA | | | |
| SNP131 | 2 | AATAGAAATGGAAATTGTACTC | [T/C] | 47996412 | SEQ ID |
| (solcap_ | | CATTTTGACGAATTTTGGTTGCA | | | NO: 11 |
| snp_sl_ | | CATTG[T/C]TAGGGACTTCACAA | | | |
| 36189) | | CATTGATTTGCTACTCATTTTTAT | | | |
| | | GTGCCCGTTACT | | | |
| SNP155 | 2 | ATCCTCTAATCGTTCGGCCGATT | [T/G] | 48455087 | SEQ ID |
| (solcap_ | | CGACTCAGTCGAAAAATCCTTTT | | | NO: 12 |
| snp_sl_ | | CAGT[T/G]GCTGCTTCCACCGAT | | | |
| 20063) | | GCAGATTCCTCCACCGCCACCAC | | | |
| | | CACCGCCGCCTC | | | |
| SNP151 | 2 | AAGGCGAAAATTGGGAAGGAAT | [T/C] | 48370810 | SEQ ID |
| (solcap_ | | CATTACTAGAATTTGGCCAAACA | | | NO: 13 |
| snp_sl_ | | CAAAG[T/C]ACCTTGATGTGATA | | | |
| 21966) | | GTCACAGGTGCAATGGCTCAAT | | | |
| | | ATATACCTACTTTG | | | |
| SNP193 | 2 | ATTTTGTTTCTTTTGATTCAATTT | [A/G] | 48602171 | SEQ ID |
| (solcap_ | | CAGTTTCTTCTTTTCTCCTTTTCA | | | NO: 14 |
| snp_sl_ | | CA[A/G]AAGCAAAAAGACAGAG | | | |
| 58447) | | AACAGCTGCAATCAAGATCAAT | | | |
| | | GTTCCAACTACA | | | |
| SNP097 | 2 | TTCTTCAATTGTTGGTCTGAAAT | [T/C] | 48697164 | SEQ ID |
| (solcap_ | | AGCGCGCAGTGGAGATTCCAAT | | | NO: 15 |
| snp_sl_ | | CTTAT[T/C]GTTAATAAACCGCCG | | | |
| 20049) | | GTTCTGGACCGTTGGTTTCCTGA | | | |
| | | ATTTGTTGCTAG | | | |
| SNP034 | 2 | GGTAACAAAATATACGTAGAAG | [A/G] | 47948927 | SEQ ID |
| (solcap_ | | GGGGATATTACTTCGAGGGGTAT | | | NO: 16 |
| snp_sl_ | | TGGGT[A/G]GAAATCGTGGGAAC | | | |
| 21867) | | GTACGATAACATCACCGACAGG | | | |
| | | TATTTAGCAATAGA | | | |

The following were used for Linkage construction
556 SNPs used for linkage construction
330 SNPs were non-redundant (not co-segregated)
Mapped to 12 linkage groups
Linkage groups ranged from 79.2 cM to 174.9 cM
Total length 1,567.2 cM
Based on physical coordinates, linkage groups covered most of the genome

As indicted in Table 1, based on chromosome positions as indicated in the tomato genome version SL 2.40, said S. lycopersicum plant is characterized by the presence of least one of the following variants:
(a) variant A or T of SNP solcap_snp_sl_1815 at position 74315042 of chromosome 1 on the tomato genome version 2.40 (SEQ ID NO: 1)
(b) variant C or G of SNP solcap_snp_sl_1819 at position 74329911 of chromosome 1 on the tomato genome version 2.40 (SEQ ID NO: 2)
(c) variant A or T of SNP solcap_snp_sl_1824 at position 74464953 of chromosome 1 on the tomato genome version 2.40 (SEQ ID NO: 3)
(d) variant A or G of SNP solcap_snp_sl_17075 at position 74483269 of chromosome 1 on the tomato genome version 2.40 (SEQ ID NO: 4)
(e) variant A or T of SNP solcap_snp_sl_1827 at position 74602284 of chromosome 1 on the tomato genome version 2.40 (SEQ ID NO: 5)
(f) variant A or G of SNP solcap_snp_sl_21280 at position 76783695 of chromosome 1 on the tomato genome version 2.40 (SEQ ID NO: 6)
(g) variant A or C of SNP solcap_snp_sl_34568 at position 76905658 of chromosome 1 on the tomato genome version 2.40 (SEQ ID NO: 7)
(h) variant T or C of SNP solcap_snp_sl_36202 at position 47651366 of chromosome 2 on the tomato genome version 2.40 (SEQ ID NO: 8)
(i) variant A or G of SNP solcap_snp_sl_21866 at position 47950096 of chromosome 2 on the tomato genome version 2.40 (SEQ ID NO: 9)
(j) variant A or G of SNP solcap_snp_sl_21862 at position 47981517 of chromosome 2 on the tomato genome version 2.40 (SEQ ID NO: 10)
(k) variant T or C of SNP solcap_snp_sl_31689 at position 47996412 of chromosome 2 on the tomato genome version 2.40 (SEQ ID NO: 11)
(l) variant T or G of SNP solcap_snp_sl_20063 at position 48455087 of chromosome 2 on the tomato genome version 2.40 (SEQ ID NO: 12)
(m) variant T or C of SNP solcap_snp_sl_21966 at position 48370810 of chromosome 2 on the tomato genome version 2.40 (SEQ ID NO: 13)
(n) variant A or G of SNP solcap_snp_sl_58447 at position 48602171 of chromosome 2 on the tomato genome version 2.40 (SEQ ID NO: 14)
(o) variant T or C of SNP solcap_snp_sl_20049 at position 48697164 of chromosome 2 on the tomato genome version 2.40 (SEQ ID NO: 15)
(p) variant A or G of SNP solcap_snp_sl_21867 at position 47948927 of chromosome 2 on the tomato genome version 2.40 (SEQ ID NO: 16)

The above-identified markers were subsequently validated for their presence in the progeny plants. It was found that the markers are associated with GBNV-resistance trait.

### Example 4

### Production of TOSPO Virus Resistant Tomato Line by Non-Transgenic Method

The BC2F6 progenies, resulting from individual plant selections (pedigree selection) since when the BC2F3 families were first screened in the rainy season in Hyderabad have shown high resistance levels to TOSPO during Kharif 16 at the hot spot locations. A summary data on the TOSPO scores of these progenies in K 16 vs checks and historically in their parent families are shown in Table 2 below.

TABLE 2
TOSPO Resistant lines at BC2F6 vs susceptible (SP 1 & 2) parents, Resistant
(RP) parent and a general susceptible check along with historical TOSPO scores over seasons
TOSPO scores over seasons

| | | | | TOSPO | | TOSPO | |
|---|---|---|---|---|---|---|---|
| | | TOSPO | K 16 | Scr | | Scr K | |
| | | Scr | Fruit | K 16 (Lost | TOSPO | 15 | TOSPO |
| Kharif(K)16 | Source | K 16 | Wt | due | Scr | Fruit | Scr |
| code | # in K15 | MEAN ± SD | (g) | Flood) | K15 | Wt (g) | K14 |
| GB0001 | H2-7-2-1 | 4.69 ± 1.11 | 30 | 4.21 ± 0.97 | 3 ± 1.87 | 15 | 3.71 ± 143 |
| GB0024 | H17-5-1 | 4.83 ± 041 | 25 | 3.67 ± 1.12 | 1.6 ± 1.01 | 31 | 2.50 ± 1.46 |
| GB0027 | GB0049-1-SB | 4.75 ± 0.45 | 35 | 3.75 ± 0.71 | 5 ± 0 | 20 | 4.10 ± 1.16 |
| GB0038 | GB0720-1-2 | 4.36 ± 0.67 | 120 | 4 ± 0.76 | 5 ± 0 | | |
| GB0039 | GB0279-2-1 | 4.31 ± 0.48 | 50 | 4.15 ± 0.69 | 4.25 ± 1.50 | 50 | |
| SP1 | SP1 | 1.5 ± 0.22 | 60 | 1.6 ± 0.2 | 1.2 ± 0.18 | 55 | 1.5 ± 0.15 |
| SP2 | SP2 | 1.6 ± 0.18 | 65 | 1.3 ± 0.5 | 1.4 ± 0.28 | 60 | 1.6 ± 0.2 |
| RP | RP | 5 ± 0 | 5 | 4.9 ± 0.22 | 4.9 ± 0.24 | 5 | 4.8 ± 0.15 |
| GBE002 | GB0134-1 | 3.77 ± 1.17 | 80 | 4.27 ± 0.65 | 4 ± 0 | 15 | 4 |
| GBE003 | GB0134-3 | 4.73 ± 0.47 | 60 | 4 ± 0.77 | 4 ± 0 | 15 | 4 |
| GBE004 | GB0135-1 | 4 ± 1.11 | 20 | 4.80 ± 0.45 | 4.5 ± 0.57 | 20 | 4 |
| GBE009 | GB0202-4 | 3.91 ± 0.70 | 35 | 3.6 ± 1.12 | 5 ± 0 | 23 | 4 |
| GBE034 | GB0074-1 | 4.69 ± 0.48 | 100 | 4.07 ± 1.14 | 1.57 ± 1.51 | | 5 |
| GBE035 | GB0074-2 | 4.29 ± 0.47 | 90 | 4.27 ± 1.19 | 1.57 ± 1.51 | | 5 |
| GBE036 | GB0075-1 | 3.50 ± 1.07 | 35 | 5 ± 0 | 4 ± 2 | 15 | 5 |
| GBE040 | GB0196-1 | 4.46 ± 0.52 | 150 | 4 ± 1.58 | 1 ± 0 | | 4 |
| GBE062 | GB0703-2 | 3.88 ± 1.81 | 120 | 3.79 ± 1.72 | 5 ± 0 | | 5 |
| GBE064 | GB0704-1 | 4.64 ± 0.50 | 120 | 3.92 ± 1.31 | | | 5 |
| GBE073 | GB0708-1 | 4.86 ± 0.36 | 100 | 4.21 ± 0.97 | 4 ± 0 | | 5 |
| GBE074 | GB0709-1 | 4.62 ± 0.51 | 150 | 4.43 ± 1.09 | | 5 | 5 |
| GBE075 | GB0710-1 | 4.64 ± 0.50 | 120 | 3.50 ± 0.84 | 5 ± 0 | | 5 |
| GBE083 | GB0782-1 | 4.55 ± 0.69 | 90 | 3.67 ± 0.50 | 5 ± 0 | | 5 |
| GBE084 | GB0782-BK | 4.83 ± 0.39 | 30 | 4.46 ± 0.66 | 5 ± 0 | | 5 |
| GBE085 | GB0783-1 | 4.85 ± 0.38 | 90 | 3.93 ± 1.21 | | | 5 |
| GBE086 | GB0783-BK | 4.90 ± 0.32 | 30 | 4.33 ± 0.98 | | | 5 |
| GBE087 | GB0784-BK | 4.08 ± 1.44 | 25 | 4.80 ± 0.42 | | | 5 |
| GBE088 | GB0785-BK | 4.43 ± 0.51 | 25 | 4 ± 0.67 | | | 5 |
| SEL-4 (S | SEL-4 | 1.71 ± 0.49 | 50 | 1.8 ± 0.56 | 2 ± 0.47 | | 1.7 ± 0.56 |
| check) | | | | | | | |

In FIG. 4 a few TOSPO resistant lines in the field vs susceptible checks are presented.

### Example 5

### Production of TOSPO Virus Resistant Tomato Line by Marker Assisted Breeding

Following table show the results of SNP marker analysis showing linkage with the TOSPO resistance trait.

TABLE 3
SNP association with resistance at 40 or 60 days

| | SNP (as | | | | | | | | | |
|---|---|---|---|---|---|---|---|---|---|---|
| | indicated in | | | | | | | | | |
| | SoICAP Infinium | Mean Resistance | | | | | | | | |
| | genotyping SNP | Index | | | F-Test | | | T-Test | | |
| | panel) | A | B | H | A/B | A/H | B/H | A/B | A/H | B/H |
| QT | (solcap_snp_sl_1815) | 4.01 | 4.44 | 3.85 | 0.0237 | 0.6354 | 0.0164 | 1E−06 | 0.1729 | 3E−07 |
| L1 | | (209) | (209) | (117) | | | | | | |
| | solcap_snp_sl_1819 | 4.01 | 4.43 | 3.83 | 0.0153 | 0.6749 | 0.0145 | 7E−07 | 0.1248 | 1E−07 |
| | | (215) | (215) | (116) | | | | | | |
| | solcap_snp_sl_1824 | 3.91 | 4.52 | 3.92 | 2E−07 | 0.6044 | 0.001 | 8E−14 | 0.9208 | 4E−07 |
| | | (267) | (267) | (103) | | | | | | |
| | solcap_snp_sl_17075 | 3.98 | 4.47 | 3.82 | 0.0064 | 0.2713 | 0.0008 | 2E−09 | 0.1754 | 7E−08 |
| | | (239) | (239) | (110) | | | | | | |
| | solcap_snp_sl_1827 | 3.82 | 4.51 | 3.79 | 1E−05 | 0.2667 | 3E-05 | 3E−17 | 0.8699 | 2E−06 |
| | | (242) | (242) | (73) | | | | | | |
| | solcap_snp_sl_21280 | 3.89 | 4.53 | 3.7 | 2E−08 | 0.2066 | 2E-07 | 8E−17 | 0.3148 | 4E−07 |
| | | (260) | (260) | (71) | | | | | | |
| QT | solcap_snp_sl_36202 | 3.52 | 4.56 | 4.04 | 2E−15 | 0.0534 | 6E-14 | 3E−30 | 0.0034 | 0.0019 |
| L2 | | (223) | (223) | (47) | | | | | | |
| | solcap_snp_sl_21866 | 3.5 | 4.56 | 4.13 | 3E−15 | 0.4657 | 0.0004 | 5E−31 | 0.0004 | 0.0057 |
| | | (222) | (222) | (48) | | | | | | |
| | solcap_snp_sl_21862 | 3.5 | 4.56 | 4.13 | 3E−15 | 0.4657 | 0.0004 | 5E−31 | 0.0004 | 0.0057 |
| | | (222) | (222) | (48) | | | | | | |
| | solcap_snp_sl_36189 | 3.51 | 4.56 | 4.18 | 3E−15 | 0.3954 | 0.0004 | 2E−30 | 0.0001 | 0.0111 |
| | | (225) | (225) | (51) | | | | | | |
| | solcap_snp_sl_20063 | 3.71 | 4.53 | 4.26 | 1E−15 | 0.1956 | 0.001 | 2E−22 | 0.0006 | 0.0474 |
| | | (264) | (264) | (61) | | | | | | |
| | solcap_snp_sl_21966 | 3.71 | 4.53 | 4.25 | 2E−15 | 0.2413 | 0.0011 | 3E−22 | 0.0013 | 0.0432 |
| | | (263) | (263) | (57) | | | | | | |
| | solcap_snp_sl_58447 | 3.69 | 4.53 | 4.2 | 3E−16 | 0.1546 | 0.0007 | 1E−22 | 0.0012 | 0.0128 |
| | | (254) | (254) | (64) | | | | | | |
| | solcap_snp_sl_20049 | 3.69 | 4.53 | 4.2 | 3E−16 | 0.1546 | 0.0007 | 1E−22 | 0.0012 | 0.0128 |
| | | (254) | (254) | (64) | | | | | | |
| | solcap_snp_sl_21867 | 3.5 | 4.56 | 4.13 | 2E−15 | 0.4657 | 0.0003 | 4E−31 | 0.0004 | 0.0056 |
| | | (222) | (222) | (48) | | | | | | |

TABLE 4
Association of QTLs with index resistance

| | | Mean | | | | | | | | |
|---|---|---|---|---|---|---|---|---|---|---|
| | | Resistance | | | | | | | | |
| Random | | Index | | | F-Test | | | T-Test | | |
| SNP | SNP | A | B | H | A/B | A/H | B/H | A/B | A/H | B/H |
| | SNP506 | 3.62 | 2.96 | 3.48 | 0.2265 | 0.2554 | 0.8112 | 0.0448 | 0.5999 | 0.2201 |
| | | (23) | (8) | (14) | | | | | | |
| | SNP507 | 3.32 | 3.53 | 3.6 | 0.4254 | 0.9966 | 0.4276 | 0.5264 | 0.3218 | 0.8441 |
| | | (16) | (12) | (16) | | | | | | |
| | SNP508 | 3.38 | 3.41 | 3.59 | 0.9498 | 0.3004 | 0.2685 | 0.9134 | 0.4894 | 0.5577 |
| | | (16) | (18) | (11) | | | | | | |

SNP flanking the QTL1 and QTL2 were significantly associated with resistance index
Random SNP were not associated with the resistance index
Table 5 provides the impact of either or both QTLs

| | | F-test | | | T-test | | |
|---|---|---|---|---|---|---|---|
| | Resistance | | | QTL1/ | | | QTL1/ |
| QTL | index | QTL1 | QTL2 | QTL2 | QTL1 | QTL2 | QTL2 |
| — | 3.59 (86) | 0.124189557 | 0.356191085 | 1.11696E−07 | 0.029388201 | 0.001159854 | 6.99241E−14 |
| QTL1 | 3.24 (129) | | 0.007012633 | 5.22526E−18 | | 1.55039E−08 | 2.58405E−24 |
| QTL2 | 4.06 (112) | | | 9.24693E−06 | | | 5.14217E−08 |
| QTL1/QTL2 | 4.61 (438) | | | | | | |

Lines carrying QTL2 had a significantly higher resistance index than the lines carrying QTL1
Lines carrying both QTL had significantly higher resistance index than either QTL

The results indicate that the inventors were successful in the development of Solanum lycopersicum progeny tomato plant which exhibits resistance to Groundnut bud necrosis virus infection.

1. A method of producing a tomato plant, wherein the tomato plant exhibits resistance to Groundnut bud necrosis virus infection, comprising:
i) providing a donor Solanum peruvianum tomato plant exhibiting resistance to Groundnut bud necrosis virus infection, wherein the donor plant comprises at least one SNP corresponding to a quantitative trait locus (QTL) indicative of Groundnut bud necrosis virus resistance on chromosome 1 or chromosome 2, and wherein the SNP is selected from a group consisting of:
(a) variant A or T of SNP solcap_snp_sl_1815 at position 74315042 of chromosome 1 on the tomato genome version 2.40 (SEQ ID NO: 1);
(b) variant C or G of SNP solcap_snp_sl_1819 at position 74329911 of chromosome 1 on the tomato genome version 2.40 (SEQ ID NO: 2);
(c) variant A or T of SNP solcap_snp_sl_1824 at position 74464953 of chromosome 1 on the tomato genome version 2.40 (SEQ ID NO: 3);
(d) variant A or G of SNP solcap_snp_sl_17075 at position 74483269 of chromosome 1 on the tomato genome version 2.40 (SEQ ID NO: 4);
(e) variant A or T of SNP solcap_snp_sl_1827 at position 74602284 of chromosome 1 on the tomato genome version 2.40 (SEQ ID NO: 5);
(f) variant A or G of SNP solcap_snp_sl_21280 at position 76783695 of chromosome 1 on the tomato genome version 2.40 (SEQ ID NO: 6);
(g) variant A or C of SNP solcap_snp_sl_34568 at position 76905658 of chromosome 1 on the tomato genome version 2.40 (SEQ ID NO: 7);
(h) variant T or C of SNP solcap_snp_sl_36202 at position 47651366 of chromosome 2 on the tomato genome version 2.40 (SEQ ID NO: 8);
(i) variant A or G of SNP solcap_snp_sl_21866 at position 47950096 of chromosome 2 on the tomato genome version 2.40 (SEQ ID NO: 9);
(j) variant A or G of SNP solcap_snp_sl_21862 at position 47981517 of chromosome 2 on the tomato genome version 2.40 (SEQ ID NO: 10);
(k) variant T or C of SNP solcap_snp_sl_31689 at position 47996412 of chromosome 2 on the tomato genome version 2.40 (SEQ ID NO: 11);
(l) variant T or G of SNP solcap_snp_sl_20063 at position 48455087 of chromosome 2 on the tomato genome version 2.40 (SEQ ID NO: 12);
(m)variant T or C of SNP solcap_snp_sl_21966 at position 48370810 of chromosome 2 on the tomato genome version 2.40 (SEQ ID NO: 13);
(n) variant A or G of SNP solcap_snp_sl_58447 at position 48602171 of chromosome 2 on the tomato genome version 2.40 (SEQ ID NO: 14);
(o) variant T or C of SNP solcap_snp_sl_20049 at position 48697164 of chromosome 2 on the tomato genome version 2.40 (SEQ ID NO: 15); and
(p) variant A or G of SNP solcap_snp_sl_21867 at position 47948927 of chromosome 2 on the tomato genome version 2.40 (SEQ ID NO: 16);
ii) crossing the donor tomato plant with a recipient Solanum lycopersicum plant to produce F1 Solanum lycopersicum progeny tomato plants, wherein the recipient tomato plants does not exhibit resistance to Groundnut bud necrosis virus and the crossing results in introgression of one or more quantitative trait locus (QTL) on chromosome 1 or chromosome 2 indicative of Groundnut bud necrosis virus resistance in the progeny tomato plants;
iii) generating further offspring progeny tomato plants from said F1 progeny plant by backcrossing or selfing; and
iv) identifying and selecting a Groundnut bud necrosis virus resistant progeny tomato plant obtained through step (b) and step (c), wherein the progeny tomato plant comprises at least one SNP corresponding to a quantitative trait locus (QTL) indicative of Groundnut bud necrosis virus resistance on chromosome 1 or chromosome 2, and wherein the SNP is selected from a group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

2. The method for producing the tomato plant of claim 1, wherein the donor tomato plant is Solanum peruvianum AVRDC accession number L00671.

3. The method for producing the tomato plant of claim 1, wherein SNPs present on chromosome 1 are indicated by SEQ ID NO: 1, SEQ ID NO: 2, SEQ ID NO: 3, SEQ ID NO: 4, SEQ ID NO: 5, SEQ ID NO: 6 and SEQ ID NO: 7 and SNPs present on chromosome 2 are indicated by SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

4. The method for producing the tomato plant of claim 1, wherein the progeny tomato plant does not exhibit one or more trait indicative of Groundnut bud necrosis virus infection selected from a group consisting of deformed shape, uneven ripening, yellow patch formation on the fruit, wilting of the top portion of the plant, dark brown spots on the leaves and dark brown patches on stems of the plants.

5. A method of identifying a tomato plant resistant to Groundnut bud necrosis virus infection, comprising identifying through genotyping in a nucleic acid sample of the tomato plant at least one SNP selected from a group consisting of:
(a) variant A or T of SNP solcap_snp_sl_1815 at position 74315042 of chromosome 1 on the tomato genome version 2.40 (SEQ ID NO: 1);
(b) variant C or G of SNP solcap_snp_sl_1819 at position 74329911 of chromosome 1 on the tomato genome version 2.40 (SEQ ID NO: 2);
(c) variant A or T of SNP solcap_snp_sl_1824 at position 74464953 of chromosome 1 on the tomato genome version 2.40 (SEQ ID NO: 3);
(d) variant A or G of SNP solcap_snp_sl_17075 at position 74483269 of chromosome 1 on the tomato genome version 2.40 (SEQ ID NO: 4);
(e) variant A or T of SNP solcap_snp_sl_1827 at position 74602284 of chromosome 1 on the tomato genome version 2.40 (SEQ ID NO: 5);
(f) variant A or G of SNP solcap_snp_sl_21280 at position 76783695 of chromosome 1 on the tomato genome version 2.40 (SEQ ID NO: 6);
(g) variant A or C of SNP solcap_snp_sl_34568 at position 76905658 of chromosome 1 on the tomato genome version 2.40 (SEQ ID NO: 7);
(h) variant T or C of SNP solcap_snp_sl_36202 at position 47651366 of chromosome 2 on the tomato genome version 2.40 (SEQ ID NO: 8);
(i) variant A or G of SNP solcap_snp_sl_21866 at position 47950096 of chromosome 2 on the tomato genome version 2.40 (SEQ ID NO: 9);
(j) variant A or G of SNP solcap_snp_sl_21862 at position 47981517 of chromosome 2 on the tomato genome version 2.40 (SEQ ID NO: 10);
(k) variant T or C of SNP solcap_snp_sl_31689 at position 47996412 of chromosome 2 on the tomato genome version 2.40 (SEQ ID NO: 11);
(l) variant T or G of SNP solcap_snp_sl_20063 at position 48455087 of chromosome 2 on the tomato genome version 2.40 (SEQ ID NO: 12);
(m) variant T or C of SNP solcap_snp_sl_21966 at position 48370810 of chromosome 2 on the tomato genome version 2.40 (SEQ ID NO: 13);
(n) variant A or G of SNP solcap_snp_sl_58447 at position 48602171 of chromosome 2 on the tomato genome version 2.40 (SEQ ID NO: 14);
(o) variant T or C of SNP solcap_snp_sl_20049 at position 48697164 of chromosome 2 on the tomato genome version 2.40 (SEQ ID NO: 15); and
(p) variant A or G of SNP solcap_snp_sl_21867 at position 47948927 of chromosome 2 on the tomato genome version 2.40 (SEQ ID NO: 16);
wherein the presence of one or more SNP is indicative of Groundnut bud necrosis virus infection resistance trait of the tomato plant.

6. The method of selecting a tomato plant as claimed in claim 5, wherein SNPs present on chromosome 1 are indicated by SEQ ID NO: 1, SEQ ID NO: 2, SEQ ID NO: 3, SEQ ID NO: 4, SEQ ID NO: 5, SEQ ID NO: 6 and SEQ ID NO: 7 and SNPs present on chromosome 2 are indicated by SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

7. A tomato plant exhibiting resistance to Groundnut bud necrosis virus infection produced by a method of claim 1, wherein the tomato plant comprises at least one SNP corresponding to a quantitative trait locus on chromosome 1 or chromosome 2, and wherein the SNP is selected from a group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

8. The tomato plant as claimed in claim 7, wherein SNPs present on chromosome 1 are indicated by SEQ ID NO: 1, SEQ ID NO: 2, SEQ ID NO: 3, SEQ ID NO: 4, SEQ ID NO: 5, SEQ ID NO: 6 and SEQ ID NO: 7 and SNPs present on chromosome 2 are indicated by SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

9. A seed of the tomato plant as claimed in claim 6.
